(19) United States
(12) Patent Application Publication
(10) Pub. No.: US 2017/0247438 A1
(43) Pub. Date: Aug. 31, 2017

(54) SHARK ANTIBODIES FOR CANCER THERAPY

(71) Applicant: Prolume, Ltd., Pinetop, AZ (US)

(72) Inventor: Bruce Bryan, Beverly Hills, CA (US)

(21) Appl. No.: 15/596,973

(22) Filed: May 16, 2017

(51) Int. Cl. C07K 16/18; C07K 16/30

(52) U.S. Cl. CPC C07K 16/18; C07K 16/30; C07K 2317/20; C07K 2317/14

(57) ABSTRACT

Sharks have been thriving for over 500 million years. Sharks evolved some of the first adaptive antibodies and developed immunoglobulin M (IgM) as their primary and only class of circulating antibody. Laboratory research exposing sharks to carcinogens has failed to illicit cancers in these animals, and while reports of sharks bearing tumors exist, it is rare to find in nature.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### TECHNICAL FIELD

The technical field relates to antibody generation in sharks for cancer therapy.

### BACKGROUND

Cancers grow and behave in a similar manner to primordial germ line cells and rapidly dividing embryonic cells; most cancers contain both normal antigens and antigens that are tissue specific and unique to themselves.

Some early developmental antigens may be re-expressed in tumor cells, or may also be genetically mutated proteins, and occur in the developing fetus. These are known as oncological-fetal or “oncofetal” antigens.

“Complement” is a pathway of thirty (30) interacting proteins and special activating proteases working together to form a “Membrane Attack Complex”, which bores a large open hole into the cell which destroys the cell's ability to maintain sodium-potassium-chloride and anion gradients, which results in a spewing of the internal cell machinery out. Triggered by antibody binding to the cell surface, Complement can rapidly destroy any cell type once the collection of proteins is activated upon their surface.

The present invention is directed toward precisely triggering Complement to specifically kill only cancer cells or other cells we wish to remove, such as excess fat cell, excess benign prostate hypertrophy, and of course cancer cells.

### SUMMARY OF THE EMBODIMENTS

The disclosure teaches a method to immunize sharks against the individual human cancer cells taken directly from a patient during surgery or at biopsy. Then, immunizing small easily handled sharks to the cancer. Once several booster immunizations have been made, the resulting hyperimmune plasma or serum is injected back into the patient to cause lysis of their tumor cells.

The disclosure teaches a tumor cell line injected into any species of shark, for the production of immune sera from said shark(s) for therapeutic use of any kind.

The disclosure teaches an antibody or antibody fragment derived from sharks, containing a Complement binding domain, and used therapeutically in humans or animals for the lysis of unwanted cell types of any kind.

The disclosure teaches hybridomas created for the purpose of manufacturing shark antibodies of any kind.

Various modifications and additions can be made to the embodiments discussed without departing from the scope of the invention. For example, while the embodiments described above refer to particular features, the scope of this invention also included embodiments having different combination of features and embodiments that do not include all of the above described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is pictorial description of complement on spanning the cell membrane.

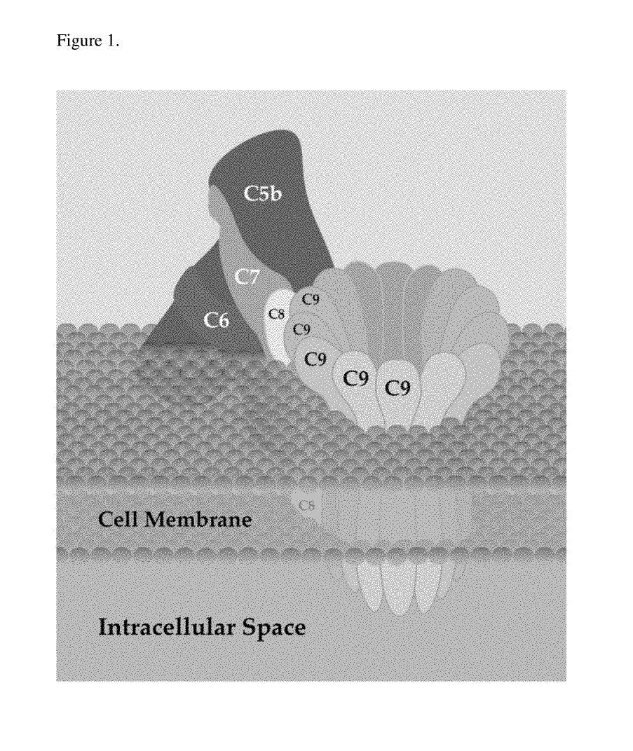

### DETAILED DESCRIPTION

While various aspects and features of certain embodiments have been summarized above, the following detailed description illustrates a few embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described and claimed herein, and while various features are ascribed to different embodiments, it should be appreciated that the features described with respect to one embodiment may be incorporated with other embodiments as well. By the same token, however, no single feature or features of any described or claimed embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

Any type of mammalian cell can be injected into sharks for the purpose of developing within the shark an antibody collection that will recognize and bind to that cell type, or purified antigenic components of any heterologous protein or protein carbohydrate moiety.

Ways of boosting antibody responses are well known to those in the art, such as the addition of haptens (EG: Dinitrophenols, Urushiols, etc.) and or adjuvants that contain elements that prolong the antigen exposure to the host such as Freund's Complete or Incomplete adjuvant.

Repeated “booster” injections of foreign antigens and/or foreign cells into sharks will greatly amplify their titer of antibody and stimulate circulating antibody producing cells. Usually a three-week boosting period is sufficient but can be altered by using tropical sharks at higher ambient temperatures if antibodies are desired sooner.

Using antigens affixed to stationary phases (EG. Agarose or Sepharose beads) cells or antibodies are purified from shark blood and used by those skilled in the art to “clone-out” the antibody producing cells and/or the antibody directly.

Libraries of the shark antibodies are made, and then the library is selected using a human tumor line, or directly clone from mRNA, shark antibody producing cells, or by using molecular biologic means (EG: LOX or CRISPR) rapidly devise techniques to target specific genes for recovery and subsequent commercial production of shark antibodies or their active regions, and make targeting mini-antibody (EG “Minibody” or “Minibodies,”), or make active Fragments of antibody that bind antigens of commercial value, that are effective against human tumors, hypertrophic tissues, or in medical diagnosis.

Fusion proteins of antibodies are made to target specific antigens on tumor cells using fluorescent analogs, fluorescent proteins, or bioluminescent luciferase fusions for observing, locating, or defining the anatomical location of tumors.

Hybridomas are made for the production of monoclonal shark antibodies for a particular use or function, not limited to, but easily manufactured and used to de-bulk tumor masses, reduce fatty cell accumulations by localized injections, for reduction of organ size such as in benign or prostatic hypertrophy, and obliteration with surveillance to maintain permanent remission of metastatic human or animal cancers.

Since shark immune systems cannot distinguish between normal and human cancer antigens; subtractive molecular biologic or solid substrate absorption schemes are constructed to remove shark antibodies or monoclonal hybridoma products, (or other clones created by any other means) designed for the production of specific antibodies that would otherwise be contaminated with “innocent bystander” antibody.

Removal of innocent bystander antibody may be required in some instances where specific purified antibody was unavailable, or when heterologous mixtures of antibodies might cause a rapid flux of potassium or serum sickness, after therapeutic administration of shark antibody compositions; resulting from “innocent bystander” cross reactions.

Shark antibody itself is used for a specific Hapten (used herein in the immunological meaning) to create an immune response that would not otherwise occur in humans or animals.

### EXAMPLES

The following examples are provided for illustrative purposes only and are not intended to limit the scope of the invention.

### Example 1

What was discovered was several antibody types, mainly the new more modern evolutionary antibodies, as found in modern mammals are made up of many subtypes adapted for specific functions in various tissue types. So the concept of excess antigen shedding into the bloodstream by tumor cells was shown. These large numbers of floating antigens bind all the circulating antibodies and immune cell receptors; creating the “antigen excess” theory.

Another concept of “antibody blocking” was shown. It involves covering the tissue with the host's own antibodies; antibodies that do not activate Complement, and lymphocyte policeman pass by the cell, “seeing” the cell as covered with the tail end of antibodies recognized as “self” and do not mount a cellular response. This is much like Tubercle bacillus that covers itself with a kind of wax, making it difficult for cell receptors and antibodies to directly kill the cells, so the response by the organism is to end up walling the foreign body off.

Since higher animals have many subtypes of antibodies, and many cell types; and cancer remains despite an immune response, what's needed is an antibody that did not know about all the complexity of modern immunity, and antibody that just bound to the cell, triggered the Complement to drill a hole, and kill the cancer or fat cell.

Primitive shark antibodies respond and kill rat red blood cells.

Some small sharks were immunized to rat red blood cells and the serum was shown to destroy the red cells which was very easy to detect with the unaided eye, hemoglobin being soluble turned the test tube water red and clear when the red cells were broken.

Heating the shark serum to 56° C. for 30 minutes,; the blood cells were no longer broken open the Complement was inactivated.

Of the five classes of antibodies, IgM (the first to evolve), IgG, IgA, IgD and IgE., sharks only make two types of IgM, a monomeric and pentameric form composed of the five of the monomers joined at the center like a starfish.

Most importantly is the pentameric form is extremely efficient at activating complement because as the ends of the antibody bind the antigen the bend and open a hidden Complement binding site that is just perfect for one molecule of the first Complement of protein to bridge and activate building of the Membrane Attack Complex on the cell next to the a IgM site.

Human serum Complement was used to bust open cells activated by Shark IgM antibody. Rat Red Blood Cell shark serum that was heated (to destroy Complement activity) was added to Rat Red Blood Cells in saline solution, no lysis was observed until Human Complement was added, which immediately resulted in rapid and complete turning of the saline water red and clear.

This was repeated for every mammalian complement available in the lab, included Guinea Pig, Mouse, Rabbit, and purchased serum from Cows. Hyper immunized Shark serum against a Rat Fibrosarcoma prevents or remove the tumor.

The description of the various embodiments has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiments described and shown in the FIGURE were chosen and described in order to explain the principles of the invention, the practical application, and to enable others of ordinary skill in the art to understand the invention for various embodiments with various modifications as are suited to the particular use contemplated. All references cited herein are incorporated in their entirety by reference.

What is claimed is:

1. A tumor cell line injected into any species of shark, for the production of immune sera from said shark(s) for therapeutic use of any kind.

2. An antibody or antibody fragment derived from sharks, containing a Complement binding domain, and used therapeutically in humans or animals for the lysis of unwanted cell types of any kind.

3. Hybridomas created for the purpose of manufacturing shark antibodies of any kind.
